(19) United States
(12) Patent Application Publication
(10) Pub. No.: US 2011/0313303 A1
(43) Pub. Date: Dec. 22, 2011

(54) Autonomic Nervous Balance Computation Apparatus and Method Therefor

(75) Inventor: Mayumi Higa, Shiga (JP)

(73) Assignee: CHAOS TECHNOLOGY RESEARCH LABORATORY, Shiga (JP)

(21) Appl. No.: 13/125,670

(22) PCT Filed: Mar. 10, 2010
(86) PCT No.: PCT/JP2010/001684

§ 371 Date: Apr. 22, 2011

(30) Foreign Application Priority Data
Mar. 10, 2009 (JP) 2009-056003

(51) Int. Cl. A61B 5/02

(52) U.S. Cl. 600/500

(57) ABSTRACT

End pulse wave measurement means 3 measures an end pulse wave. Heart rate computation means 16 computes a heart rate per unit time from the measured end pulse wave. Autonomic nervous balance computation means 17 defines the heart rate per unit time as HR, and calculates an approximate value HFa of the autonomic nervous balance by Equation (1) below:

HFa=k1*HR³+k2*HR²+k3*HR+k4 Equation (1)

where k1=−0.0003, k2=0.0796, k3=−8.5795, and k4=325.3. This makes it possible to obtain autonomic nervous balance from the heart rate HR. Hence, the autonomic nervous balance is determined through measurement for a short period.

### TECHNICAL FIELD

The present invention relates to an autonomic nervous balance computation apparatus, and in particular to the shortening of a measurement period.

### BACKGROUND ART

Evaluation of autonomic nervous balance is determined in accordance with the balance between parasympathetic nerve activity (HF) and sympathetic nerve activity (LF). Higher parasympathetic nerve activity indicates a relaxed state, and higher sympathetic nerve activity indicates a tense state.

Evaluation of autonomic nervous balance that uses a pulse wave according to the related art is described briefly. HF and LF are calculated from a pulse wave, and the interval of peaks in the pulse wave is measured. The interval of peaks in a pulse wave, which is considered to be constant in a resting state, is actually not constant but slightly fluctuated. Thus, a waveform that represents such fluctuations is calculated, and a spectrum is calculated from the waveform. The values of HF and LF are calculated by calculating the area of a specific portion in the spectrum. In general, it is necessary that these computations should be performed on the basis of the results of measurement for five minutes or more.

### RELATED ART DOCUMENT

### Patent Document

Patent Document 1: JP-A-2004-358022

### SUMMARY OF THE INVENTION

### Problem to be Solved by the Invention

If measurement of autonomic nervous balance based on HF and LF obtained from a pulse wave is performed in home or the like, a measurement period of five minutes is long. The inventor conducted a variety of experiments, and had an impression that a measurement of about 2.5 minutes could provide some precision. However, a measurement period of 2.5 minutes is still long for home use.

In the case where the measurement period is long, several problems arise. (1) Variations in autonomic nerve activity and variations in biological state often occur in a short period, e.g., less than 2.5 minutes, and thus such variations cannot be recorded. (2) The homeostasis of a biological state cannot be secured, and the homeostasis conditions for the analysis of LF and HF are not met. (3) A large measurement error, which tends to interfere with noise such as disturbance and body motion, occurs. (4) Inconveniently, a subject cannot stay still for a long period during measurement performed in situ.

In order to solve the foregoing problems, it is an object of the present invention to provide an autonomic nervous balance computation apparatus and method that can calculate autonomic nervous balance in a short period. A further object of the present invention is to provide an autonomic nervous balance computation apparatus and method that considers blood vessel balance.

The characteristics, other objects, usages, effects, and so forth of the present invention will become apparent by referencing the embodiment and the drawings.

### Means for Solving the Problem

(1) The present invention provides an autonomic nervous balance computation apparatus including: 1) heart rate computation means for computing a heart rate per unit time from a measured end pulse wave; and 2) autonomic nervous balance computation means for computing autonomic nervous balance on the basis of the heart rate per unit time, in which 3) defining the heart rate per unit time as HR, the autonomic nervous balance computation means calculates an approximate value HFa of the autonomic nervous balance by Equation (1) below:

HFa=k1*HR³+k2*HR²+k3*HR+k4 Equation (1)

where k1, k2, k3, and k4 are constants.

Thus, an approximate value of the autonomic nervous balance can be calculated in a short period.

(2) In the autonomic nervous balance computation apparatus according to the present invention, k1=−0.0003, k2=0.0796, k3=−8.5795, and k4=325.3, and k1 to k4 have an increase/reduction range of 0.9 to 1.1 times with respect to the respective specified value. Thus, an approximate value of the autonomic nervous balance can be calculated in a short period.

(3) The autonomic nervous balance computation apparatus according to the present invention further includes:

wave height value computation means for calculating wave height values of an early-systolic-period positive wave, an early-systolic-period negative wave, and a later-systolic-period re-descending wave for an acceleration pulse wave obtained from the measured end pulse wave;

first wave height ratio computation means for calculating an absolute value of a value obtained by dividing a sum of the wave height values of the early-systolic-period negative wave and the later-systolic-period re-descending wave by the wave height value of the early-systolic-period positive wave as a first wave height ratio;

second wave height ratio computation means for calculating an absolute value of a value obtained by dividing a difference between the wave height values of the early-systolic-period negative wave and the later-systolic-period re-descending wave by the wave height value of the early-systolic-period positive wave as a second wave height ratio;

second threshold storage means for storing a second threshold for the second wave height ratio;

blood vessel balance index computation means for calculating a blood vessel balance index Pb by Equation (1) below in the case where the second wave height ratio is less than the second threshold and by Equation (2) below in the case where the second wave height ratio is not less than the second threshold:

Pb=k11*(first wave height ratio)+α 1)

Pb=k12*(second wave height ratio)+β (12)

where k11, k12, α, and β are constants; and

autonomic nervous balance determination means for determining the autonomic nervous balance in consideration of blood vessel balance on the basis of the blood vessel balance index Pb and the approximate autonomic nervous balance HFa.

By placing focus on the value of the second wave height ratio to calculate the blood vessel balance index on the basis of the value of the first wave height ratio in the case where the second wave height ratio is less than the second threshold and on the basis of the value of the second wave height ratio in the case where the second wave height ratio is not less than the second threshold in this way, it is possible to eliminate fluctuations in blood vessel balance index or the like in the case where noise is mixed in the obtained value of the second wave height ratio. Thus, the blood vessel balance can be determined precisely even through measurement for a relatively short period. This also makes it possible to determine the autonomic nervous balance in consideration of the blood vessel balance.

(4) The autonomic nervous balance computation apparatus according to the present invention further includes:

wave height value computation means for calculating wave height values of an early-systolic-period positive wave, an early-systolic-period negative wave, and a later-systolic-period re-descending wave for an acceleration pulse wave obtained from the measured end pulse wave;

first wave height ratio computation means for calculating an absolute value of a value obtained by dividing a sum of the wave height values of the early-systolic-period negative wave and the later-systolic-period re-descending wave by the wave height value of the early-systolic-period positive wave as a first wave height ratio;

second wave height ratio computation means for calculating an absolute value of a value obtained by dividing a difference between the wave height values of the early-systolic-period negative wave and the later-systolic-period re-descending wave by the wave height values of the early-systolic-period positive wave as a second wave height ratio;

first threshold storage means for storing a first threshold for the first wave height ratio;

blood vessel balance index computation means for calculating a blood vessel balance index Pb by Equation (1) below in the case where the first wave height ratio is not less than the first threshold and by Equation (2) below in the case where the first wave height ratio is less than the first threshold:

Pb=k1*(first wave height ratio)+α (1)

Pb=k2*(second wave height ratio)+β (2)

where k1, α, and β are constants; and

autonomic nervous balance determination means for determining the autonomic nervous balance in consideration of blood vessel balance on the basis of the blood vessel balance index Pb and the approximate autonomic nervous balance HFa.

In this way, by placing focus on the value of the first wave height ratio to calculate the blood vessel balance index on the basis of the value of the first wave height ratio in the case where the first wave height ratio is not less than the first threshold and on the basis of the value of the second wave height ratio in the case where the first wave height ratio is less than the first threshold, it is possible to eliminate fluctuations in blood vessel balance index or the like in the case where noise is mixed in the obtained value of the first wave height ratio. Thus, precise determination can be made even through measurement for a relatively short period. This also makes it possible to determine the autonomic nervous balance in consideration of the blood vessel balance.

(5) The autonomic nervous balance computation apparatus according to the present invention further includes acceleration pulse wave computation means for calculating an acceleration pulse wave from the measured fingertip pulse wave. Thus, an acceleration pulse wave can be obtained.

(6) The autonomic nervous balance computation apparatus according to the present invention further includes end pulse wave measurement means for measuring an end pulse wave. Thus, an end pulse wave can be measured.

(7) The present invention also provides an autonomic nervous balance computation method including the steps of: computing a heart rate per unit time from a measured end pulse wave; and computing autonomic nervous balance on the basis of the heart rate per unit time, in which defining the heart rate per unit time as HR, an approximate value HFa of the autonomic nervous balance is calculated by Equation (1) below:

HFa=k1*HR³+k2*HR²+k3*HR+k4 Equation (1)

where k1, k2, k3, and k4 are constants.

Thus, an approximate value of the autonomic nervous balance can be calculated in a short period.

The term “autonomic nervous balance” as used herein refers to the state of the balance of autonomic nerves determined by the balance between parasympathetic nerve activity (HF) and sympathetic nerve activity (LF).

The term “blood vessel balance” corresponds to the concept representing the functionality of blood vessels, and means the elasticity and plasticity of the blood vessels. In the embodiment, the acceleration pulse wave computation means 5 is implemented by the CPU 23 and the process in step S3 of FIG. 4.

a wave to e wave shown in FIG. 3C are called a-wave (early-systolic-period positive wave), b-wave (early-systolic-period negative wave), c-wave (middle-systolic-period re-ascending wave), d-wave (later-systolic-period re-descending wave), and e-wave (early-diastolic-period positive wave).

### BRIEF DESCRIPTION OF THE DRAWINGS

FIG. 1 is a functional block diagram of an autonomic nervous balance computation apparatus 1 according to an embodiment of the present invention.

FIG. 2 shows the hardware configuration of the autonomic nervous balance computation apparatus 1 in the case where the autonomic nervous balance computation apparatus 1 is implemented using a CPU.

FIG. 3 shows examples of a measured pulse wave and an acceleration pulse wave.

FIG. 4 is a flowchart of a determination program.

FIG. 5 is a graph showing the relationship between the heart rate and HF (un).

FIG. 6 is a graph showing the relationship between the heart rate and HF (un).

FIG. 7 shows examples of the acceleration pulse wave.

FIG. 8 is a detailed flowchart for calculation of blood vessel balance performed in the determination program.

### EMBODIMENT OF THE INVENTION

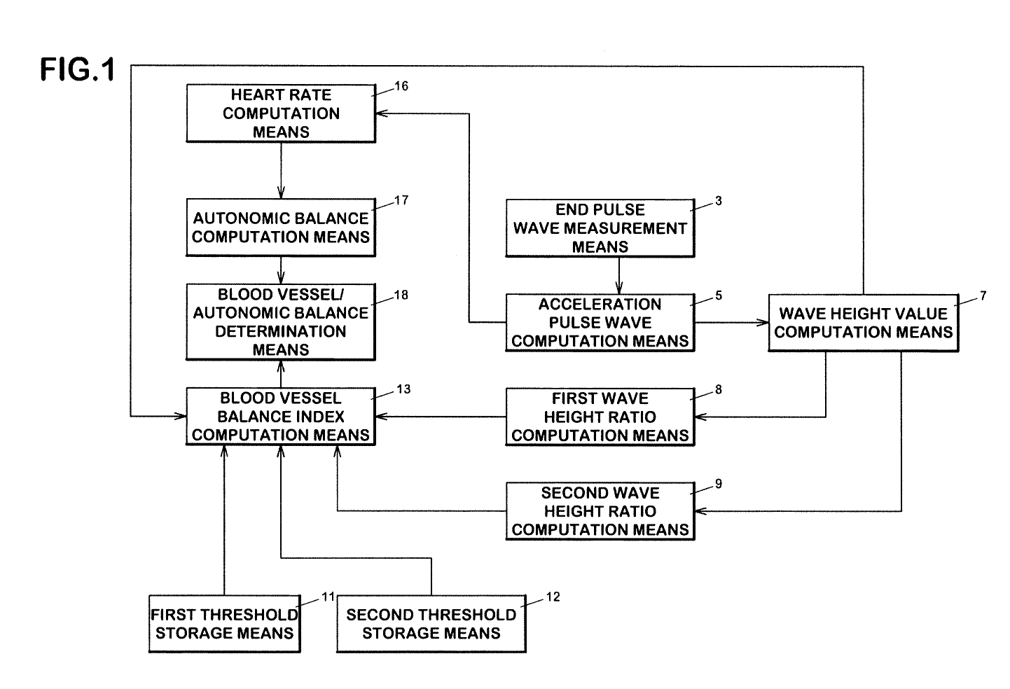

FIG. 1 is a functional block diagram of an autonomic nervous balance computation apparatus according to an embodiment of the present invention.

An autonomic nervous balance computation apparatus 1 includes end pulse wave measurement means 3, acceleration pulse wave computation means 5, wave height value computation means 7, first wave height ratio computation means 8, second wave height ratio computation means 9, first threshold storage means 11, second threshold storage means 12, blood vessel balance index computation means 13, heart rate computation means 16, autonomic nervous balance computation means 17, and blood vessel/autonomic nervous balance determination means 18.

The end pulse wave measurement means 3 measures an end pulse wave. The heart rate computation means 16 computes a heart rate per unit time from the measured end pulse wave. The autonomic nervous balance computation means 17 defines the heart rate per unit time as HR, and calculates an approximate value HFa of the autonomic nervous balance by Equation (1) below.

HFa=k1*HR³+k2*HR²+k3*HR+k4 Equation (1)

where k1=−0.0003, k2=0.0796, k3=−8.5795, and k4=325.3, and k1 to k4 are allowed to vary within an increase/reduction range of 0.9 to 1.1 times with respect to the respective specified values.

This makes it possible to obtain autonomic nervous balance from the heart rate HR.

The acceleration pulse wave computation means 5 calculates an acceleration pulse wave from the measured end pulse wave. The wave height value computation means 7 calculates wave height values of an early-systolic-period positive wave, an early-systolic-period negative wave, and a later-systolic-period re-descending wave for the acceleration pulse wave obtained from the measured end pulse wave. The first wave height ratio computation means 8 calculates the absolute value of a value obtained by dividing the sum of the wave height values of the early-systolic-period negative wave and the later-systolic-period re-descending wave by the wave height value of the early-systolic-period positive wave as a first wave height ratio. The second wave height ratio computation means 9 calculates the absolute value of a value obtained by dividing the difference between the wave height values of the early-systolic-period negative wave and the later-systolic-period re-descending wave by the wave height value of the early-systolic-period positive wave as a second wave height ratio.

The first threshold storage means 11 stores a first threshold for the first wave height ratio. The second threshold storage means 12 stores a second threshold for the second wave height ratio.

The blood vessel balance index computation means 13 calculates a blood vessel balance index Pb by Equation (1) below in the case where the second wave height ratio is less than the second threshold and by Equation (2) below in the case where the second wave height ratio is not less than the second threshold.

Pb=k1*(first wave height ratio)+α (1)

Pb=k2*(second wave height ratio)+β (2)

where k1, α, and β are constants.

By placing focus on the value of the second wave height ratio to calculate the blood vessel balance index on the basis of the value of the first wave height ratio in the case where the second wave height ratio is less than the second threshold and on the basis of the value of the second wave height ratio in the case where the second wave height ratio is not less than the second threshold in this way, it is possible to eliminate fluctuations in blood vessel balance index or the like in the case where noise is mixed in the obtained value of the second wave height ratio. Thus, the blood vessel balance can be determined precisely even through measurement for a relatively short period.

The blood vessel/autonomic nervous balance determination means 18 determines autonomic nervous balance in consideration of the blood vessel balance from the blood vessel balance index Pb and the approximate autonomic nervous balance HFa. This makes it possible to compute the autonomic nervous balance in consideration of the blood vessel balance.

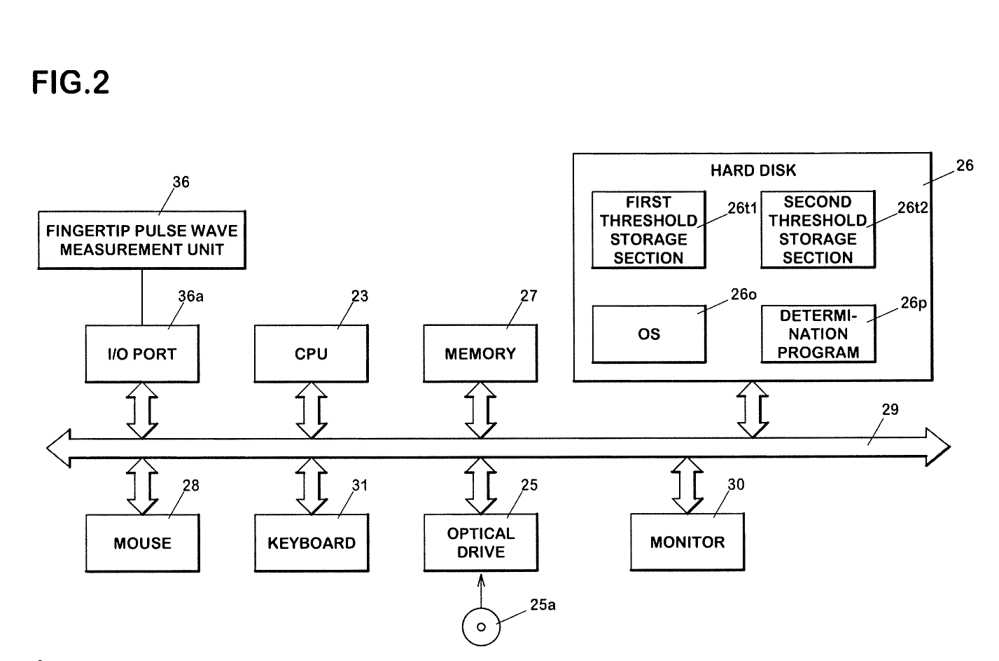

Next, the hardware configuration of the autonomic nervous balance computation apparatus 1 will be described. FIG. 2 shows an example of the hardware configuration of the autonomic nervous balance computation apparatus 1 implemented using a CPU.

The autonomic nervous balance computation apparatus 1 includes a CPU 23, a memory 27, a hard disk 26, a monitor 30, an optical drive 25, a mouse 28, a keyboard 31, an I/O port 36a, and a bus line 29. The CPU 23 controls the various sections via the bus line 29 in accordance with various programs stored in the hard disk 26.

The hard disk 26 has an operating system program (hereinafter abbreviated as an OS) 26o and a determination program 26p.

A fingertip pulse wave measurement unit 36 is connected to the I/O port 36a. The fingertip pulse wave measurement unit 36 is a common fingertip pulse wave measurement unit. In the embodiment, a fingertip pulse wave measurement unit that measures a blood flow rate using infrared light to calculate a fingertip pulse wave from the blood flow rate is adopted. Specifically, a light emitting element emits infrared light, which is reflected by a subject finger so that the reflected infrared light is received by a light receiving element. The intensity of the reflected light indicates the blood flow rate. Thus, a signal output from the light receiving element indicates a fingertip plethysmogram. The signal from the light receiving element is converted into digital data to be output.

The data from the fingertip pulse wave measurement unit 36 are taken into the CPU 23 via the I/O port 36a.

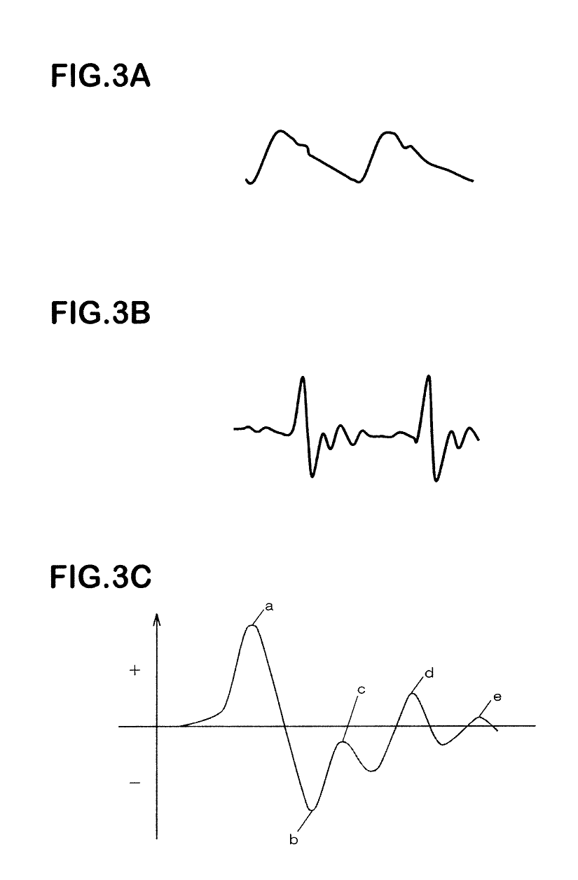

FIG. 3A shows an example of the fingertip pulse wave output from the fingertip pulse wave measurement unit 36. While the fingertip pulse wave is actually digital data, the fingertip pulse wave is shown as a waveform in the drawing.

A first threshold storage section 26t1 and a second threshold storage section 26t2 store thresholds for the first wave height ratio and the second wave height ratio, respectively, as discussed later.

The process performed by the determination program 26p will be discussed in detail later. While linux (registered trademark or trademark) is adopted as the operating system program (OS) 26o in the embodiment, the present invention is not limited thereto.

Each of the programs described above is read via the optical drive 25 from a CD-ROM 25a, in which the program is stored, and installed on the hard disk 26. The program may be installed on the hard disk from a computer-readable storage medium such as a flexible disk (FD), an IC card, or the like besides the CD-ROM. Further, the program may be downloaded using a communication line.

In the embodiment, the program stored in the CD-ROM is indirectly executed by a computer, with the program installed on the hard disk 26 from the CD-ROM. However, the present invention is not limited thereto, and the program stored in the CD-ROM may be directly executed from the optical drive 25. The program that can be executed by the computer includes a program that can be directly executed after installation, a program that can be executed after conversion into other forms (such as decompression of compressed data, for example), and a program that can be executed by combination with other module portions.

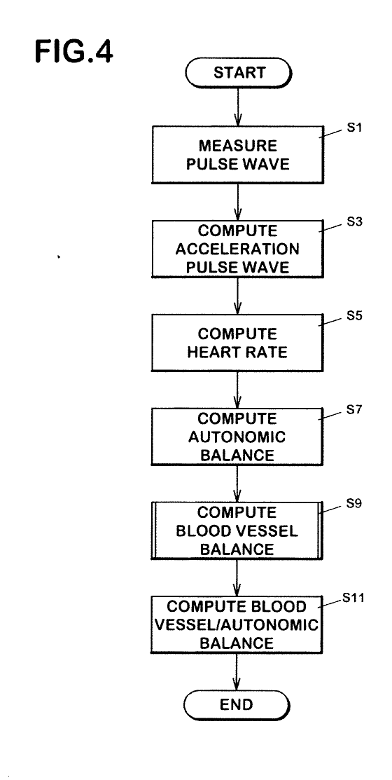

The process performed by the CPU 23 in accordance with the determination program 26p will be described with reference to FIG. 4.

The CPU 23 gives a command to measure a pulse wave to the fingertip pulse wave measurement unit 36 to measure a pulse wave (step S1 of FIG. 4). A measured pulse wave from the fingertip pulse wave measurement unit 36 is stored in the memory 27.

The CPU 23 computes an acceleration pulse wave from the pulse wave stored in the memory 27 (step S3 of FIG. 4). The acceleration pulse wave is obtained by differentiating the measured pulse wave twice in the same way as in the related art. The acceleration pulse wave is shown in FIG. 3B.

The CPU 23 computes a heart rate per unit time from the acceleration pulse wave. In the embodiment, peaks in the acceleration pulse wave shown in FIG. 3B for 30 seconds are counted as an average heart rate to calculate a heart rate HR for one minute.

The CPU 23 calculates an approximate value HFa of the autonomic nervous balance by Equation (1) below on the basis of the heart rate HR for one minute (step S7).

HFa=k1*HR³+k2*HR²+k3*HR+k4 Equation (1)

where k1=−0.0003, k2=0.0796, k3=−8.5795, and k4=325.3.

The relationship between the approximate value HFa and the autonomic nervous balance will be described.

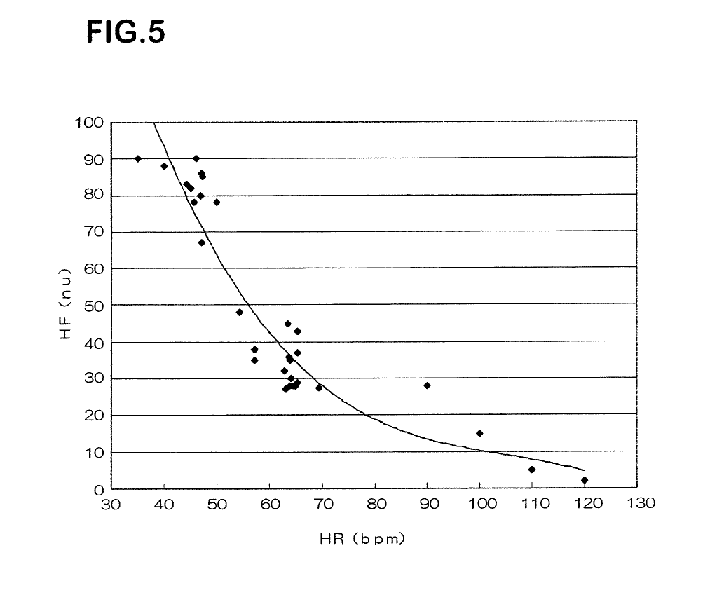

In the related art, as described already, autonomic nervous balance is determined in accordance with the balance between parasympathetic nerve activity (HF) and sympathetic nerve activity (LF). In contrast, the inventor considered that there is a correlation between the heart rate and the autonomic nervous balance, and conducted experiments to find such a correlation. FIG. 5 shows the results of experiments conducted on 30 people. In FIG. 5, the horizontal axis represents HR (heart rate (beats/minute)) calculated on the basis of data for a short period of 30 seconds, and the vertical axis represents the value of HF (un) (in %) (normalized HF). The normalized HF (un) is calculated by Equation (2) below on the basis of HF and LF calculated by a method according to the related art (measurement for five minutes).

HF(un)=HF/(HF+LF) (2)

As shown in the drawing, HR and HF (un) can be approximated to each other by Equation (1) which corresponds to a cubit regression curve. Hence, the approximate value HFa calculated by Equation (I) can be assumed as HF (un).

A determination coefficient for Equation (1) is obtained as a determination coefficient R²=0.8871, which is sufficient for a value that can be measured in a short period.

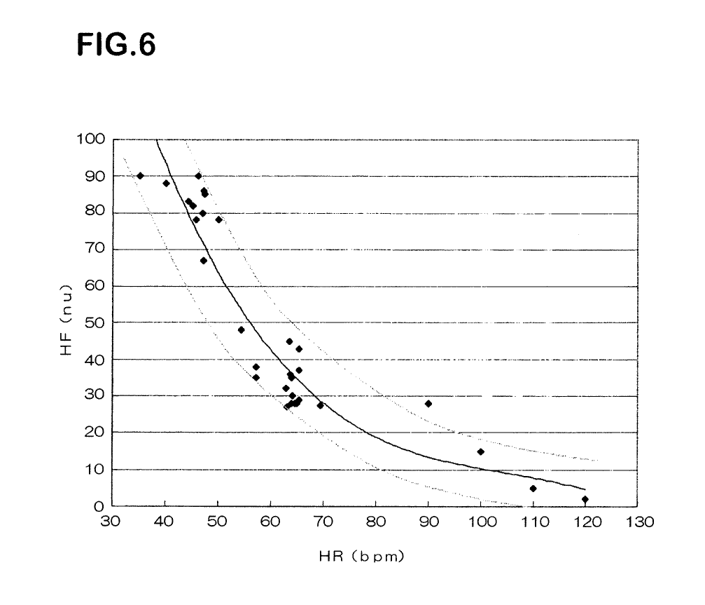

For Equation (1), k1 to k4 are allowed to vary within an increase/reduction range of 0.9 to 1.1 times with respect to the respective specified value, which causes the determination coefficient R² to become substantially 1. An approximated curve in the case where k1 to k4 are varied is shown in FIG. 6.

This allows the approximate value HFa to be represented by 0 to 100. In this case, an HFa of 40 to 60 indicates that the autonomic nervous balance is normal, an HFa of 0 to 39 indicates that the sympathetic nerves are dominant, and an HFa of 61 to 100 indicates that the parasympathetic nerves are dominant.

By computing an approximate value of the autonomic nervous balance on the basis of the heart rate by Equation (1) in this way, precise determination can be made even through measurement for a relatively short period.

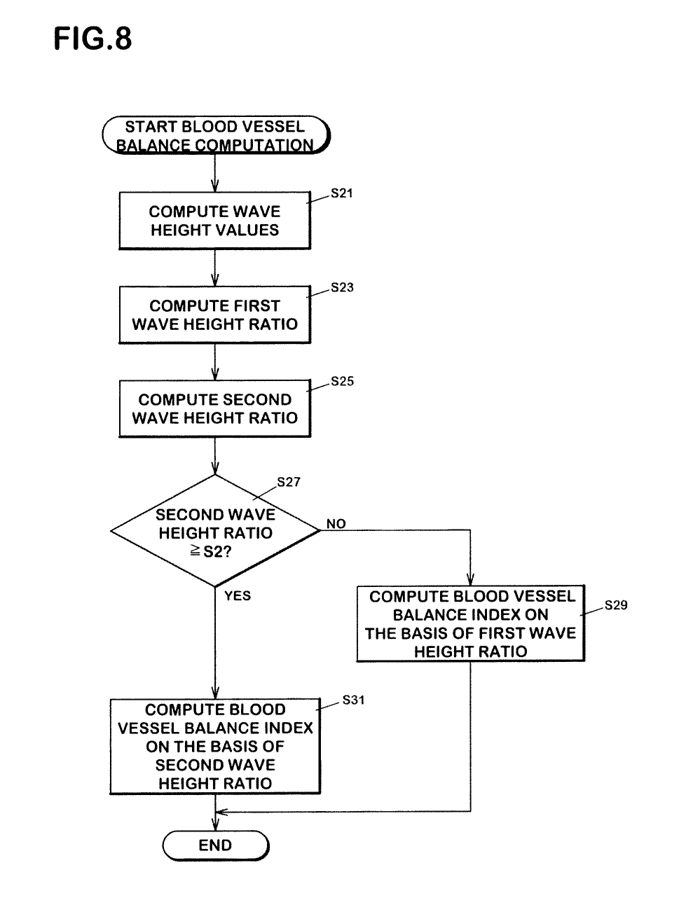

Next, the CPU 23 computes a blood vessel balance index (step S9 of FIG. 4). The computation of the blood vessel balance index will be described with reference to FIG. 8.

First, the CPU 23 computes wave height values (step S21 of FIG. 8). The wave height values are described. The acceleration pulse wave shown in FIG. 3C has a-wave, b-wave, c-wave, d-wave, and e-wave. In the embodiment, a-wave, b-wave, and d-wave are used as discussed later, and thus the values of such waves are calculated. The CPU 23 computes a first wave height ratio (step S23 of FIG. 8). The first wave height ratio is a value obtained by dividing the absolute value of the sum of the value of b wave and the value of d wave by the value of a wave. The CPU 23 computes a second wave height ratio (step S25 of FIG. 8). The second wave height ratio is a value obtained by dividing the absolute value of the difference between the value of b-wave and the value of d-wave by the value of a-wave. The wave height values of b wave and d wave are both negative. The absolute values of (b−d) and (d−b), however, result in the same value.

The CPU 23 reads the second threshold S2 stored in the wave height value of the second threshold storage section t2 to compare the second threshold S2 with the second wave height ratio calculated in step S7 (S27 of FIG. 8). -{ }-Then, in the case where the second wave height ratio is less than the threshold S2, a blood vessel balance index is computed on the basis of the first wave height ratio (step S29).

In the embodiment, the blood vessel balance index Pb in this case is computed by Equation (11) below.

Pb=k11*(first wave height ratio)+α (11)

where S2=0.25, k11=90, and α=135.

In contrast, in the case where the second wave height ratio is not less than the threshold S2 in step S27 of FIG. 8, a blood vessel balance index Pb is computed on the basis of the second wave height ratio (step S31).

In the embodiment, the blood vessel balance index in this case is computed by Equation (12) below.

Pb=k12*(second wave height ratio)+β (12)

where S2=0.25, k12=40, and β=31.

The blood vessel balance index Pb is computed in this way.

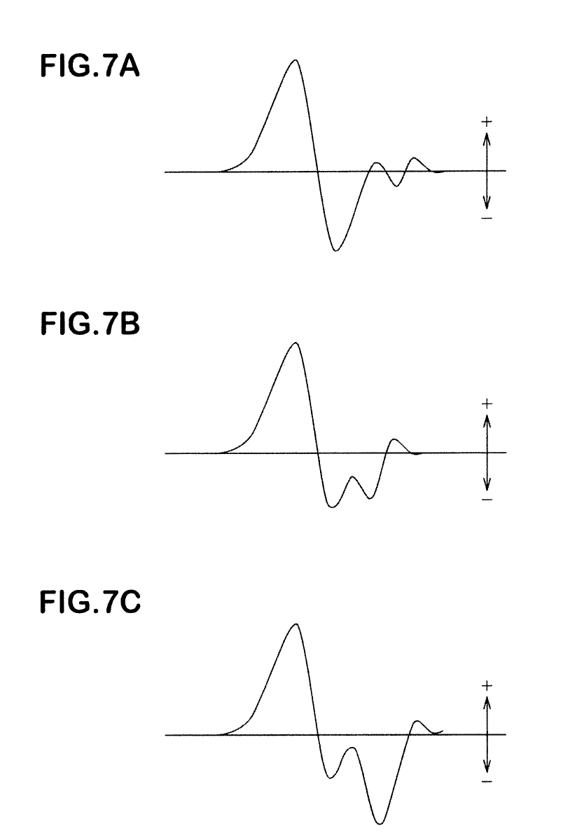

This enables precise determination even in the case where there is little difference between the value of b-wave and the value of d-wave as in FIG. 7B.

The blood vessel balance index Pb may be finally normalized by Equation (13) below.

Normalized blood vessel balance index Ps=−(Pb−actual age) (13)

a) If Ps is less than −5, unbalanced (the blood vessels tend to be hardened).

b) If Ps is not less than −5 but less than +5, the blood vessel balance is normal.

c) If Ps is not less than +5, unbalanced (the blood vessels tend to be plastically deformed).

Thus, by selectively using Equation (11) and Equation (12), precise determination can be made not only in the case where there is a difference between the value of b-wave and the value of d-wave as shown in FIGS. 7A and 7C, but also in the case where there is little difference between the value of b-wave and the value of d-wave as shown in FIG. 7B. Thus, the blood vessel balance can be quantitatively estimated more precisely even under a measurement environment with much noise.

Next, the CPU 23 computes blood vessel/autonomic nervous balance (step S11 of FIG. 4).

In the embodiment, both the normalized blood vessel balance index Ps and the approximate value HFa are evaluated in three grades. The former is evaluated as “over-hardened blood vessels”, “normal blood vessel balance”, and “over-plasticized blood vessels”, and the latter is evaluated as “dominant parasympathetic nerves”, “normal autonomic nervous balance”, and “dominant sympathetic nerves”.

Thus, by combining these evaluations, the blood vessel/autonomic nervous balance may be indicated as “over-hardened blood vessels/dominant parasympathetic nerves”, for example. By indicating the blood vessel/autonomic nervous balance obtained by combining the blood vessel balance and the autonomic nervous balance in this way, it is possible to conveniently acquire information on the autonomic nervous balance and the blood vessel balance, which are correlated to each other.

For example, if “over-hardened blood vessels/dominant parasympathetic nerves” is indicated, it is found that the hardening of the blood vessels is caused by the imbalance of the autonomic nerves, and vice versa. Meanwhile, if “over-hardened blood vessels/normal autonomic nervous balance” is indicated, it is found that the hardening of the blood vessels is caused by aging.

In the embodiment, the fingertip pulse wave measurement unit 36 is provided in the autonomic nervous balance computation apparatus 1. However, a fingertip pulse wave measurement unit may be connected to a communication device (such as a cellular phone, for example) to transmit a calculated acceleration pulse wave to a center computer, which may return the results of calculation performed by the center computer to the communication device. Thus, the functions of the autonomic nervous balance computation apparatus 1 may be provided by a plurality of devices, not by a single device.

The acceleration pulse wave computation means may be provided in the center computer, not in the fingertip pulse wave measurement unit. In the case where the functions of the autonomic nervous balance computation apparatus 1 are provided by a plurality of devices in this way, any configuration that is functionally possible may be used.

In the embodiment, a fingertip pulse wave is adopted as the end pulse wave. However, other peripheral pulse waves such as a foot pulse wave may also be used.

In the embodiment, focus is placed on the difference between the values of b-wave and d-wave. However, focus may be placed on the sum of the values of b-wave and d-wave. That is, in the case where the value of the first wave height ratio is not less than a predetermined value, it may be determined that the value of the first wave height ratio is not significantly affected by noise to calculate the blood vessel balance index on the basis of the first wave height ratio. On the other hand, in the case where the value of the first wave height ratio is less than the predetermined value, it may be determined that the value of the first wave height ratio is significantly affected by noise to calculate the blood vessel balance index on the basis of the second wave height ratio.

In the embodiment, the constants S1, k11, α, k12, and β in Equations (11) and (12) are defined as described above. However, the present invention is not limited thereto.

The acceleration pulse wave computation means may be provided in the center computer, not in the fingertip pulse wave measurement unit. In the case where the functions of the autonomic nervous balance computation apparatus 1 are provided by a plurality of devices in this way, any configuration that is functionally possible may be used.

In the embodiment, the absolute values sum of the first wave height ratio and the second wave height ratio are used. However, the absolute values may be obtained by an absolute value (ie, neglecting the negative sign) of the first wave height ratio and an absolute value of the second wave height ratio.

The heart rate computation means 16 computes the heart rate using data on the acceleration pulse wave computed by the acceleration pulse wave computation means 5. However, the heart rate computation means 16 may compute the heart rate from the end pulse wave measured by the end pulse wave measurement means 3.

The disclosure in the above embodiment may be understood as an autonomic nervous balance computation apparatus that does not have a blood vessel balance computation function, or as a blood vessel balance computation function that does not have an autonomic nervous balance computation function.

In the above embodiment, the various functions are implemented through software using a CPU. However, some or all of the functions may be implemented through hardware such as a logic circuit.

Some of the processes of the above program may be performed by the operating system (OS).

While the present invention has been described above by way of a preferred embodiment, the various terms used in the description are not limitative but illustrative, and may be modified within the scope of the appended claims without departing from the scope and sprit of the present invention.

1. An autonomic nervous balance computation apparatus comprising:
heart rate computation means for computing a heart rate per unit time from a measured end pulse wave; and
autonomic nervous balance computation means for computing autonomic nervous balance on the basis of the heart rate per unit time,
wherein defining the heart rate per unit time as HR, the autonomic nervous balance computation means calculates an approximate value HFa of the autonomic nervous balance by Equation (1) below:
HFa=k1*HR³+k2*HR²+k3*HR+k4 Equation (1)
in which k1, k2, k3, and k4 are constants.

2. The autonomic nervous balance computation apparatus according to claim 1,
wherein k1=−0.0003, k2=0.0796, k3=−8.5795, and k4=325.3, and
k1 to k4 have an increase/reduction range of 0.9 to 1.1 times with respect to the respective specified value.

3. The autonomic nervous balance computation apparatus according to claim 2, further comprising:
wave height value computation means for calculating wave height values of an early-systolic-period positive wave, an early-systolic-period negative wave, and a later-systolic-period re-descending wave for an acceleration pulse wave obtained from the measured end pulse wave;
first wave height ratio computation means for calculating an absolute value of a value obtained by dividing a sum of the wave height values of the early-systolic-period negative wave and the later-systolic-period re-descending wave by the wave height value of the early-systolic-period positive wave as a first wave height ratio;
second wave height ratio computation means for calculating an absolute value of a value obtained by dividing a difference between the wave height values of the early-systolic-period negative wave and the later-systolic-period re-descending wave by the wave height value of the early-systolic-period positive wave as a second wave height ratio;
second threshold storage means for storing a second threshold for the second wave height ratio;
blood vessel balance index computation means for calculating a blood vessel balance index Pb by Equation (1) below in the case where the second wave height ratio is less than the second threshold and by Equation (2) below in the case where the second wave height ratio is not less than the second threshold:
Pb=k11*(first wave height ratio)+α (1)
Pb=k12*(second wave height ratio)+β (2)
in which k11, k12, α, and β are constants; and
autonomic nervous balance determination means for determining the autonomic nervous balance in consideration of blood vessel balance on the basis of the blood vessel balance index Pb and the approximate autonomic nervous balance HFa.

4. The autonomic nervous balance computation apparatus according to claim 2, further comprising:
wave height value computation means for calculating wave height values of an early-systolic-period positive wave, an early-systolic-period negative wave, and a later-systolic-period re-descending wave for an acceleration pulse wave obtained from the measured end pulse wave;
first wave height ratio computation means for calculating an absolute value of a value obtained by dividing a sum of the wave height values of the early-systolic-period negative wave and the later-systolic-period re-descending wave by the wave height value of the early-systolic-period positive wave as a first wave height ratio;
second wave height ratio computation means for calculating an absolute value of a value obtained by dividing a difference between the wave height values of the early-systolic-period negative wave and the later-systolic-period re-descending wave by the wave height value of the early-systolic-period positive wave as a second wave height ratio;
first threshold storage means for storing a first threshold for the first wave height ratio;
blood vessel balance index computation means for calculating a blood vessel balance index Pb by Equation (1) below in the case where the first wave height ratio is not less more than the first threshold and by Equation (2) below in the case where the first wave height ratio is less than the first threshold:
Pb=k1*(first wave height ratio)+α (1)
Pb=k2*(second wave height ratio)+β (2)
in which k1, α, and β are constants; and
autonomic nervous balance determination means for determining the autonomic nervous balance in consideration of blood vessel balance on the basis of the blood vessel balance index Pb and the approximate autonomic nervous balance HFa.

5. The autonomic nervous balance computation apparatus according to claim 4, further comprising:
acceleration pulse wave computation means for calculating an acceleration pulse wave from the measured end pulse wave.

6. The autonomic nervous balance computation apparatus according to claim 5, further comprising:
end pulse wave measurement means for measuring an end pulse wave.

7. An autonomic nervous balance computation method comprising the steps of:
computing a heart rate per unit time from a measured end pulse wave; and
computing autonomic nervous balance on the basis of the heart rate per unit time,
wherein defining the heart rate per unit time as HR, an approximate value HFa of the autonomic nervous balance is calculated by Equation (1) below:
HFa=k1*HR³+k2*HR²+k3*HR+k4 Equation (1)
in which k1, k2, k3, and k4 are constants.

8. A non-transitory recording medium storing a program that causes a computer to function as an autonomic nervous balance computation apparatus including the respective means according to claim 1.

9. The autonomic nervous balance computation apparatus according to claim 3, further comprising:
acceleration pulse wave computation means for calculating an acceleration pulse wave from the measured end pulse wave.

10. The autonomic nervous balance computation apparatus according to claim 6, further comprising:
end pulse wave measurement means for measuring an end pulse wave.
